(19) United States
(12) Patent Application Publication
(10) Pub. No.: US 2014/0276494 A1
(43) Pub. Date: Sep. 18, 2014

(54) Bodily Fluid Collection Devices, Bodily Fluid Collection Systems, and Methods for Removing Bodily Fluids

(71) Applicant: Hollister Incorporated, Libertyville, IL (US)

(72) Inventors: George Cisko, Spring Grove, IL (US); Peter Visconti, Gurnee, IL (US); David McDonough, Libertyville, IL (US)

(73) Assignee: Hollister Incorporated, Libertyville, IL (US)

(21) Appl. No.: 14/213,331

(22) Filed: Mar. 14, 2014

Related U.S. Application Data
(60) Provisional application No. 61/784,796, filed on Mar. 14, 2013.

(51) Int. Cl. A61F 5/44; A61M 1/00

(52) U.S. Cl. CPC A61F 5/4404; A61M 1/0023
USPC 604/319; 604/327

(57) ABSTRACT

A bodily fluid collection device includes: (a) at least one wall that at least partially defines an interior space configured for contacting a bodily fluid, the at least one wall having at least one interior wall surface; (b) a drain port coupled with at least a first portion of the at least one wall, the drain port configured to provide fluid communication between the interior space and an exterior of the device; and (c) at least one surface feature defined by at least a portion of the at least one interior wall surface, the at least one surface feature at least partially defining one or a plurality of flow pathways in the interior space. The one or the plurality of flow pathways is configured to mitigate or prevent collapse of the device, occlusion in the device, and/or closing off of the drain port when suction is applied.

### RELATED APPLICATIONS

This application claims the priority benefit of U.S. Provisional Application No. 61/784,796, filed Mar. 14, 2013. The entire contents of the provisional application are incorporated herein by reference, except that in the event of any inconsistent disclosure or definition from the present specification, the disclosure or definition herein shall be deemed to prevail.

### TECHNICAL FIELD

The present teachings relate generally to bodily fluid management systems (e.g., bowel management systems, wound drainage management systems, fistula management systems, stoma management systems, and/or the like). In some embodiments, the present teachings relate to devices (e.g., collection pouches and/or conduits) for use in bowel management systems. In other embodiments, the present teachings relate to methods for emptying such devices.

### BACKGROUND

Conventional bowel management systems presently in use involve the retention of collected fecal matter somewhere within the components of the system itself. Fecal collectors are pouches configured to receive and contain feces obtained from a patient and, therefore, are typically placed in close proximity to the anal region of the patient. Bowel catheters divert the stool or feces from the patient to a connected collection bag via a catheter or tube. Such fecal collectors may require periodic draining, such as by a caregiver. The caregiver may assist in moving stool along the catheter tube by “milking” the tube toward the collection bag. The bowel catheter collection bag may require periodic emptying and/or replacement. The frequency of emptying and/or replacement depends on the condition and care of the patient and on the bowel catheter system design.

Most activities related to the maintenance of conventional types of bowel management products are performed manually. Maintenance and emptying (e.g., evacuating or draining) of collectors, tubes, bags, and the like is susceptible to potential stool spillage and related contamination. In addition, since conventional fecal collectors and bowel catheters have specific collection systems and their related management activities are typically performed by caregivers, conventional systems and products may involve relatively high labor and economic costs.

Additionally, bowel catheter collection bags are typically secured in prominent positions near the foot of a patient's bed. As such, the devices may be highly visible to the patient and/or the patient's visitors and represent an unsightly distraction.

Collection bags and drainage tubes for these types of products are typically made from thin films and/or flexible polymeric materials. These types of materials have a tendency to collapse upon application of suction to an interior space thereof. Depending on the design, the collapsing film or other flexible material may cover the suction inlet, thereby preventing proper drainage. As a result, additional labor-intensive activity from medical personnel, caregivers, and/or the like may be needed in order to drain a collection bag, tube, or the like.

### SUMMARY

The scope of the present invention is defined solely by the appended claims, and is not affected to any degree by the statements within this summary.

By way of introduction, a bodily fluid collection device in accordance with the present teachings includes: (a) at least one wall that at least partially defines an interior space configured for contacting a bodily fluid, the at least one wall having at least one interior wall surface; (b) a drain port coupled with at least a first portion of the at least one wall, the drain port configured to provide fluid communication between the interior space and an exterior of the device; and (c) at least one surface feature defined by at least a portion of the at least one interior wall surface, the at least one surface feature at least partially defining one or a plurality of flow pathways in the interior space. The one or the plurality of flow pathways is configured to mitigate or prevent collapse of the device, occlusion in the device, and/or closing off of the drain port when suction is applied.

A bodily fluid collection system in accordance with the present teachings includes a bodily fluid collection device of a type described herein and a suction canister configured for detachable engagement with the bodily fluid collection device.

A method in accordance with the present teachings for removing a bodily fluid from a bodily fluid collection device in a bodily fluid management system includes: (a) accumulating a bodily fluid in a bodily fluid collection device of a type described herein; (b) connecting the bodily fluid collection device to a suction canister; (c) applying suction to the bodily fluid collection device; and (d) removing at least a portion of the accumulated bodily fluid from the bodily fluid collection device.

### BRIEF DESCRIPTION OF THE DRAWINGS

In the drawings, like reference characters are used to designate like elements.

FIG. 1 shows a plan view of an exemplary fecal collection pouch in accordance with the present teachings.

FIG. 2 shows a perspective view of the exemplary fecal collection pouch shown in FIG. 1.

FIG. 3 shows a partial cross-section through the line 4-4 of the exemplary fecal collection pouch shown in FIG. 1 in an expanded state.

FIG. 4 shows a partial cross-section through the line 4-4 of the exemplary fecal collection pouch shown in FIG. 1 in a collapsed state.

FIG. 5 shows a perspective view of an exemplary fecal material conduit in accordance with the present teachings.

FIG. 6 shows a plan view of an exemplary bowel management system in accordance with the present teachings.

### DETAILED DESCRIPTION

Bodily fluid collection devices, bodily fluid collection systems, and methods for removing a bodily fluid from bodily fluid collection devices (e.g., in a bowel management system, a wound drainage management system, and/or the like) have been discovered and are described herein. The devices, systems, and/or methods in accordance with the present teachings may solve and/or improve upon one or more of the above-described drawbacks or limitations associated with conventional techniques.

In accordance with the present teachings, occlusion in a bodily fluid collection device and/or system (e.g., a fecal collection device and/or system) may be minimized or eliminated by providing a network of protected channels through which suction and related drainage may be maintained. In some embodiments, the present teachings provide for an interrupted surface on one or more interior surfaces of a device that are applicable to medical collection devices (e.g., bags, pouches, and/or the like) and conduits (e.g., drainage tubes, suction tubes, and/or the like). The interrupted interior surface or surfaces may prevent total collapse of a collection device (e.g., a film pouch or drainage tube) upon application of suction and, as a result, promote the continued flow and complete drainage of material from the device into a collection or suction canister.

Throughout this description, reference is made to fecal collection devices, fecal collection systems, and methods for removing waste material from fecal collection devices as being representative and exemplary implementations in accordance with the present teachings. However, it is to be understood that such references are meant to be purely illustrative, and that the present teachings are not restricted to the collection and/or removal of fecal matter from patients. On the contrary, the present teachings are equally applicable to other types of bodily fluids in addition to or alternatively to fecal matter. By way of representative and non-limiting example, collection devices in accordance with the present teachings may be configured for receiving other bodily fluids such as urine, blood, plasma, saliva, breast milk, and/or the like, and/or bodily fluids obtained via wound drainage, fistulas, stomas, and/or the like.

It is to be further understood that elements and features of the various representative embodiments described below may be combined in different ways to produce new embodiments that likewise fall within the scope of the present teachings.

By way of general introduction, a representative fecal collection device in accordance with the present teachings is a fecal collection pouch 2 as shown in FIGS. 1, 2, 3, and 4. As best shown by the cross-sectional views in FIGS. 3 and 4, the fecal collection pouch 2 includes at least one wall 4 that at least partially defines an interior space 6 configured for contacting waste material (not shown). The at least one wall 4 comprises at least one interior wall surface 8. As best shown in FIGS. 1 and 2, the fecal collection pouch 2 further includes a drain port 10 coupled with at least a first portion 12 of the at least one wall 4. The drain port 10 is configured to provide fluid communication between the interior space 6 and an exterior of the device. The fecal collection pouch 2 further includes at least one surface feature 14 defined by at least a portion of the at least one interior wall surface 8. The at least one surface feature 14 at least partially defines one or a plurality of flow pathways 16 in the interior space 6. The one or the plurality of flow pathways 16 is configured to mitigate or prevent collapse of the device 2, occlusion in the device 2, and/or closing off of the drain port 10 when suction is applied.

As used herein, the term “coupled” is intended broadly to encompass both direct and indirect coupling. Thus, a first part is said to be coupled to a second part when the two parts are directly coupled (e.g. by direct contact or direct functional engagement), as well as when the first part is functionally engaged with an intermediate part which is in turn functionally engaged either directly or via one or more additional intermediate parts with the second part. Also, two parts are said to be coupled when they are functionally engaged (directly or indirectly) at some times and not functionally engaged at other times.

The collection bag 2 shown in FIGS. 1, 2, 3, and 4 may assist in preventing collapse of the collector walls 4 under vacuum. The principles underlying the configuration of the collection bag 2 may be used for a collection bag of a catheter system, a collection bag of a simpler fecal collector, a catheter of a bowel management system, and/or the like.

In some embodiments, the at least one wall 4 comprises a pair of opposing sidewalls 4a and 4b. As best shown by FIGS. 1 and 2, the opposing sidewalls 4a and 4b may be joined together along their respective peripheries 5a and 5b, such that they at least partially define the interior space 6. In some embodiments, one or both of the opposing walls 4a and 4b of the collector bag 2 may be formed or modified to include a surface feature 14 on an inner surface 8 thereof. The surface feature 14 may create spacing, gaps, and/or channels within the bag 2 that prevent the walls 4a and 4b from completely collapsing onto one another under vacuum. These channels may extend directly or indirectly from one end of the bag to the other and/or from one side of the bag to the other. Such a configuration may allow waste material to flow along the channels if a vacuum or suction is applied to the bag drainage outlet (e.g., drain port 10).

In some embodiments, as best shown by FIGS. 2, 3, and 4, the at least one surface feature 14 comprises one or a plurality of air bubbles 18 provided on the at least one interior wall surface 8. The one or the plurality of flow pathways 16 are defined by spaces between the one or the plurality of air bubbles 18.

In the representative examples shown in FIGS. 1, 2, 3, and 4, a fecal collector pouch 2 was created with a layer of bubble wrap anchored within the bag 2 between the film walls 4a and 4b. In some embodiments, the bubble wrap comprises polyethylene, which can be readily staked into place by heat-sealing to one edge of the fecal collector pouch 2. The product, when exposed to suction, allowed substantially complete drainage during testing since the network of channels formed between the bubbles in the wrap and the film walls remained open to the applied suction. The suction-assisting feature may be applied globally (e.g., across the complete interior surface of the product) or more locally in selected zones (e.g., in a strip down a center of the device, pouch, tube, etc.).

While bubble wrap typically has a regular pattern of air-filled pockets, it is to be understood that the same benefit may be realized using surface features having one or a plurality of irregular patterns. In addition, it is to be further understood that non-bubble wrap approaches that nonetheless provide and maintain open channels within the draining pouch or drain tube under vacuum conditions may be used with similar results. By way of example, alternate approaches may include scrim materials, embossed films, and/or the like on or for the collection device wall or walls.

The use of bubble wrap may provide one or more advantages including but not limited to the following. Since bubble wrap is soft, it is unlikely to cause patient discomfort in the event that a patient rolls onto the device during use. Bubble wrap is relatively inexpensive and readily available in many forms. Bubble wrap may be heat-sealed to pouch films. Bubble wrap may be conducive to ostomy-type “bag machine” automated manufacturing or other film handling/manufacturing methods. If one or more bubbles are inadvertently popped, the network of flow channels may still function upon application of suction. There is flexibility with respect to how to incorporate bubble wrap into a device (e.g., bubble wrap may be an applied layer on a collection device film layer or formed and integrated into or as one of the collection device walls, etc.).

In some embodiments, bubble wrap may provide a unique, clean, and high-tech appearance to fecal collection devices in accordance with the present teachings. In some embodiments, the bubble wrap may optionally be colored in order to enhance the visual appearance of the device. The aesthetic appearance may be leveraged to promote the technology across a range of products (e.g., fecal collectors, collection bags, externally applied drainage tubes, etc.).

Bubble wrap is an existing commodity product that is used as protective packaging and for insulation. In accordance with the present teachings, a heretofore untapped and potentially low-cost usage of bubble wrap to provide a key function in hospital patient care is provided.

In some embodiments, the at least one wall 4 comprises a flexible polymeric film. All manner of polymeric film is contemplated for use in accordance with the present teachings. Representative types of polymeric film include but are not limited to polyethylene, polypropylene, polyvinyl chloride, polyesters, polyurethanes, natural latex rubber, nylons, and the like, and combinations thereof. In some embodiments, the polymeric film comprises polyethylene.

In some embodiments, the volume of the interior space 6 is fixed whereas, in other embodiments, the volume of the interior space 6 may change. By way of example, when the at least one wall 4 comprises a flexible polymeric film, the interior space 6 may be configured for expansion to accommodate accumulated waste material.

In some embodiments, the fecal collection device is provided as a collection pouch 2 configured for storing waste material received from a patient, as shown for example in FIGS. 1, 2, 3, and 4. In other embodiments, the fecal collection device may be provided as a drainage tube that includes surface features, flow pathways, and/or channels on all or a portion (e.g., on half of the inner circumference) of the interior drainage tube wall. Such a drainage tube may be less susceptible to occlusion and/or collapse under vacuum conditions. By way of example, as shown in FIG. 5, the fecal collection device may be provided as a conduit 20 (e.g., a bowel drainage tube, a suction tube, and/or the like) configured for transferring waste material from a patient under vacuum. In some embodiments, the conduit 20 is substantially cylindrical. As shown in FIG. 5, the conduit 20—analogously to the collection pouch 2—includes at least one wall 22 that at least partially defines an interior space 24 configured for contacting waste material (not shown). The at least one wall 22 comprises at least one interior wall surface 26. The conduit 20 further includes at least one surface feature 28 defined by at least a portion of the at least one interior wall surface 26. The at least one surface feature 28 at least partially defines one or a plurality of flow pathways 30 in the interior space 24.

In conventional fecal collection systems, the negative pressure applied by a suction canister may collapse the drainage tube and/or walls of the collection bag upstream of the bag drain outlet. If collapse occurs, the drainage tube and/or the bag may not completely drain or empty, thereby resulting in unwanted contamination of the system, more frequent draining of the system, more frequent replacement of the drainage tube and/or bag, and/or the like. Each of these results may add to the time and financial costs associated with using the bowel management system. A fecal collection bag applied directly to the perianal region of a patient (e.g., without the intermediacy of a bowel drainage tube) is susceptible to the same type of collapsing problem and the same type of undesirable consequences as described above.

FIG. 6 shows a representative fecal collection system 32 in accordance with the present teachings. A bowel management system, such as the fecal collection system 32, may comprise a fecal collection pouch 2′ of a type described herein and a suction canister 34 configured for detachable engagement with the fecal collection pouch 2′. As shown in FIG. 6, the fecal collection pouch 2′ includes a collection inlet 36 coupled with at least a second portion 38 of the at least one wall 4. In some embodiments, as further shown in FIG. 6, the collection inlet 36 and the drain port 10 may be provided on opposing ends of the collection pouch 2′. In other embodiments, one or both of the collection inlet 36 and the drain port 10 may be provided on a face of the collection pouch 2′ (e.g., an inlet provided on the face of wound drainage collector, fistula pouch, ostomy pouch, and/or the like).

In some embodiments, a fecal collection system 32 in accordance with the present teachings further includes a suction tube 40 configured for detachable engagement with the drain port 10 of the fecal collection pouch 2′ and an inlet 42 of the suction canister 34. In some embodiments, the suction tube 40 comprises a fecal collection device in accordance with the present teachings (e.g., of a type shown, for example, in FIG. 5). In other embodiments, the suction tube 40 comprises a conventional tube (e.g., a length of vacuum tubing) able to withstand the vacuum created by suction canister 34 without completely collapsing onto itself.

In some embodiments, as further shown in FIG. 6, a fecal collection system 32 in accordance with the present teachings further includes a bowel drainage tube 44 configured for detachable engagement with the collection inlet 36 of the fecal collection pouch 2′ and an anal cavity (not shown) of the patient 46. Waste material (e.g., feces or stools) may be passed from the bowel of the patient 46 to the drainage tube 44 to a collection pouch 2′ at the other end of the drainage tube in which the waste material is collected. In some embodiments, the hospital suction canister 34 may be periodically hooked up to the drain port 10 on the collection pouch 2′. The canister 34 may be operated to apply a vacuum or suction—continuously or intermittently—to the pouch 2′ and drainage tube 44 in order to drain waste material from the drainage tube 44 and to empty the collection pouch 2′ for continued use.

In some embodiments, suction is used to move and collect a bodily fluid (e.g., feces which, in some embodiments, is present as liquid diarrhea) from a hospital patient. An externally applied fecal collector (e.g., a pouch, drainage tube, or the like) may be connected to a standard hospital suction canister for collection of liquid diarrhea. External application of the fecal collector may be achieved by using an adhesive patch that adheres to a region around the patient's anus. The distal end of the fecal collector may terminate in a fitting configured to attach to the inlet of a standard hospital suction canister. The fitting may attach to the canister either directly or, more typically, via standard hospital suction tubing. Both the suction canister and the suction tubes are standard products routinely used in hospitals. The suction tube fittings may be tapered and made from an elastomeric material to provide a snug fit on a range of suction canister lid inlet diameters.

Attachments to the suction canister may be provided through the canister lid. In some embodiments, the lid may include at least one attachment for a housing suction line, at least one attachment for the patient, and one larger diameter drain opening. The canister is drained or emptied by pouring out the collected waste material through the drain when full. In some embodiments, each of these attachments/drain openings may include a cap. Thus, the lid of the canister may be customized for an individual patient and still maintain the required vacuum level. In some embodiments, the system may be used to collect stool that is more viscous than liquid diarrhea. For example, a Y-adapter that would attach to the larger drain opening may be included with the product. The Y-adapter may be used to split the single-drain opening into two openings. One of the adapter openings may be provided for a large bore vacuum attachment (e.g., a larger diameter catheter tube), and the other adapter opening may be provided for the larger diameter drain opening.

Suction is provided and used in a wide variety of hospital settings. In some embodiments, suction may be continuous. In other embodiments, suction may be intermittent. For embodiments in which the suction is intermittent, the intermittency may occur at regular intervals and/or at random intervals. Solely by way of example, an intermittent application of suction at regular intervals may include a first interval involving vacuum (e.g., an interval 10 seconds in duration or, more generally, an interval having a duration of “x”) followed by a second interval without vacuum (e.g., an interval 15 seconds in duration or, more generally, an interval having a duration of “y”). Each patient bed may have one or more suction feeds nearby and/or one or more suction canisters and suction tubing. Thus, devices and systems in accordance with the present teachings may provide basic functionality over existing suction canister components, including but not limited to: (a) parts for attachment to a patient's body; (b) parts for attachment to suction tubing; (c) seals that are liquid tight and thus more robust for use in collecting bowel waste; and/or the like.

In some embodiments, odor associated with a filled or partially filled fecal collection pouch (or, indeed, any bodily fluid collection device in accordance with the present teachings) may be eliminated, mitigated, and/or controlled. In some embodiments, the odor may be controlled through periodic emptying of the device (e.g., by intermittent or continuous application of suction, as described above).

In some embodiments, a conduit section or a collection device portion may be pouch-like, tube-like, or have an alternative configuration. In some embodiments, a tube or pouch may be extruded or formed of film with edge seals. In some embodiments, a fecal collection device in accordance with the present teachings may include odor-barrier properties, anti-microbial coatings, and/or the like. Depending on the needs of a patient and/or caregiver, a fecal collection device in accordance with the present teachings may be provided in a pouch-like configuration with an optional extension tube configured for connecting to a suction canister. In some embodiments, the fecal collection device may include a surface feature on an interior surface thereof to keep the flow channels or pathways open and to prevent the device from occluding and/or collapsing while under suction or vacuum.

In some embodiments, the present teachings provide a product and/or system having a potentially lower cost than other presently available options, and that facilitate management by a caregiver. For example, suction canisters and suction tubing are common in hospitals and represent a relatively low-cost accessory. Moreover, many hospital staff are already fully trained in the set-up and use of a suction canister. Suction allows for “automatic” movement of waste material (e.g., liquid diarrhea) to the canister through the device conduit, thereby facilitating and simplifying management by hospital staff and/or medical personnel. The management of suction canisters (e.g., cleaning, emptying, draining, etc.) is a commonplace routine in hospital settings. Spillage associated with canister usage may be minimal and, in some embodiments, may be less than that associated with current manual draining and cleaning processes. In addition, the traditional mounting of the canister behind the patient bed provides an improved visual aesthetic for the patient and the patient's visitors.

In some embodiments, graduations may be provided on suction canisters to facilitate management of patient input and output by the caregiver. In some embodiments, the suction canister may be transparent, thereby facilitating determination of when the canister is full and ready to be emptied. In some embodiments, suction draining may be portable and, in some embodiments, may be used for patient transfers. In some embodiments, the canister may be mounted to the patient bed via standard suction canister wire frame brackets or holders. In some embodiments, passive vacuum may be applied through the use of a vacuum “grenade” or squeeze ball, which in some embodiments is a hand-held silicone bladder configured to be squeezed and attached to the system. Fecal collection devices and drainage systems in accordance with the present teachings may leverage existing products that are already commonly available in hospitals and familiar to hospital staff and medical personnel (e.g., suction canisters, suction tubing, suction bulbs, etc.), thereby simplifying use of the devices and systems and rendering their use more intuitive. By way of example, suction is already used routinely in hospital environments to manage exudates from the bodies of patients. Thus, in some embodiments, devices and systems in accordance with the present teachings may leverage known suction systems and components, thereby providing a simpler, lower cost bowl management option for the hospital and caregiver.

Suction drainage in accordance with the present teachings may involve an occlusion-resistant or occlusion-proof collector or conduit (e.g., a pouch, bag, drainage tube, and/or the like) configured for collecting fecal matter and/or other substances entering the collector or conduit from a tube. When the collector is full, it may be drained through a discharge port or drain outlet. Drainage of a fecal collection system in accordance with the present teachings may be performed using an applied vacuum or suction at the discharge port of the collector device.

In some embodiments, suction drainage may involve a separation device (e.g., an occlusion inhibitor or preventer—for example, bubble wrap or air bubbles—for one or more sidewalls of the collector). Such separation devices are configured to maintain spacing between the collector walls, thereby creating flow paths along an interior length of the collector. In the absence of such a separation device, the collector—which may have flexible Walls—is susceptible to collapse upon itself under an applied suction or vacuum. This is undesirable inasmuch as collapsed walls create pockets that are not accessible to the vacuum or suction, which may prevent complete evacuation or drainage of the collector.

A method in accordance with the present teachings for removing waste material from a fecal collection device in a bowel management system includes (a) accumulating waste material in a fecal collection device of a type described herein (e.g., fecal collection pouches 2 and/or 2′, conduit 20, and/or suction tube 40, etc.); (b) connecting the fecal collection device to a suction canister 34; (c) applying suction to the fecal collection device; and (d) removing at least a portion of the accumulated waste material from the fecal collection device.

Bowel management systems and rectal catheters of the type described herein, for which the disclosed suction drainage method and occlusion resistant fecal collectors may be useful, are disclosed in, for example, U.S. Pat. Nos. 8,323,255; 8,075,540; 7,722,583; and 7,147,627.

The entire contents of each and every patent cited herein are hereby incorporated by reference, except that in the event of any inconsistent disclosure or definition from the present specification, the disclosure or definition herein shall be deemed to prevail.

The foregoing detailed description and the accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding claim—whether independent or dependent—and that such new combinations are to be understood as forming a part of the present specification.

1. A bodily fluid collection device comprising:
at least one wall that at least partially defines an interior space configured for contacting a bodily fluid, the at least one wall comprising at least one interior wall surface;
a drain port coupled with at least a first portion of the at least one wall, the drain port configured to provide fluid communication between the interior space and an exterior of the device; and
at least one surface feature defined by at least a portion of the at least one interior wall surface, the at least one surface feature at least partially defining one or a plurality of flow pathways in the interior space;
wherein the one or the plurality of flow pathways is configured to mitigate or prevent collapse of the device, occlusion in the device, and/or closing off of the drain port when suction is applied.

2. The bodily fluid collection device of claim 1 wherein the at least one wall comprises a pair of opposing sidewalls that are joined together along their respective peripheries and that together at least partially define the interior space.

3. The bodily fluid collection device of claim 1 wherein the at least one surface feature is configured to maintain a spacing between interior wall surfaces of the device during suction, the spacing providing at least a portion of the one or the plurality of flow pathways in the interior space.

4. The bodily fluid collection device of claim 1 wherein the at least one surface feature comprises one or a plurality of air bubbles provided on the at least one interior wall surface, wherein the one or the plurality of flow pathways are defined by spaces between the one or the plurality of air bubbles.

5. The bodily fluid collection device of claim 1 wherein the interior space is configured for expansion to accommodate accumulated bodily fluid.

6. The bodily fluid collection device of claim 1 wherein the at least one wall defines a substantially cylindrical conduit configured for transferring a bodily fluid from a patient under vacuum.

7. The bodily fluid collection device of claim 1 wherein the at least one wall defines a collection pouch configured for storing a bodily fluid received from a patient.

8. The bodily fluid collection device of claim 7 further comprising a collection inlet coupled with at least a second portion of the at least one wall.

9. The bodily fluid collection device of claim 1 wherein the at least one wall comprises a flexible polymeric film.

10. The bodily fluid collection device of claim 9 wherein the flexible polymeric film is selected from the group consisting of polyethylene, polypropylene, polyvinyl chloride, polyesters, polyurethanes, natural latex rubber, nylons, and combinations thereof.

11. The bodily fluid collection device of claim 1 wherein the drain port is configured for detachable engagement with a suction tube.

12. A bodily fluid collection system comprising:
the bodily fluid collection device of claim 1; and
a suction canister configured for detachable engagement with the bodily fluid collection device.

13. The bodily fluid collection system of claim 12 further comprising a suction tube configured for detachable engagement with the drain port of the bodily fluid collection device and an inlet of the suction canister.

14. The bodily fluid collection system of claim 12 wherein the at least one wall of the bodily fluid collection device comprises a pair of opposing sidewalls that are joined together along their respective peripheries and that together at least partially define the interior space.

15. The bodily fluid collection system of claim 12 wherein the at least one surface feature is configured to maintain a spacing between interior wall surfaces of the device during suction, the spacing providing at least a portion of the one or the plurality of flow pathways in the interior space.

16. The bodily fluid collection system of claim 12 wherein the at least one surface feature of the bodily fluid collection device comprises one or a plurality of air bubbles provided on the at least one interior wall surface, and wherein the one or the plurality of flow pathways are defined by spaces between the one or the plurality of air bubbles.

17. The bodily fluid collection system of claim 11 wherein the at least one wall of the bodily fluid collection device defines a collection pouch configured for storing a bodily fluid received from a patient.

18. The bodily fluid collection system of claim 17 wherein the bodily fluid collection device further comprises a collection inlet coupled with at least a second portion of the at least one wall.

19. The bodily fluid collection system of claim 18 further comprising a bowel drainage tube configured for detachable engagement with the collection inlet of the bodily fluid collection device and an anal cavity of the patient.

20. A method for removing a bodily fluid from a bodily fluid collection device in a bodily fluid management system, the method comprising:
accumulating bodily fluid in the bodily fluid collection device of claim 1;
connecting the bodily fluid collection device to a suction canister;
applying suction to the bodily fluid collection device; and
removing at least a portion of the accumulated bodily fluid from the bodily fluid collection device.

21. The method of claim 20 wherein the bodily fluid collection device is connected to the suction canister through a suction tube configured for detachable engagement with the drain port of the bodily fluid collection device and an inlet of the suction canister.

22. The method of claim 20 wherein the at least one surface feature is configured to maintain a spacing between interior wall surfaces of the device during suction, the spacing providing at least a portion of the one or the plurality of flow pathways in the interior space.

23. The method of claim 20 wherein the at least one surface feature of the bodily fluid collection device comprises one or a plurality of air bubbles provided on the at least one interior wall surface, and wherein the one or the plurality of flow pathways are defined by spaces between the one or the plurality of air bubbles.
